(19) United States
(12) Patent Application Publication
(10) Pub. No.: US 2006/0172977 A1
(43) Pub. Date: Aug. 3, 2006

(54) Method and composition for preventing, reducing and reversing ocular ischemic neuronal damage

(76) Inventor: Gerard M Nolan, Farmington, CT (US)

Correspondence Address: Louis M Heidelberger;Reed Smith, Intellectual Property, PO Box 7990, Philadelphia, PA, 19101, US

(21) Appl. No.: 10/549,961

(22) PCT Filed: Apr. 26, 2004
(86) PCT No.: PCT/US04/13034

§ 371 Date: Sep. 16, 2005

Related U.S. Application Data
(60) Provisional application No. 60/465,476, filed on Apr. 25, 2003.

(51) Int. Cl. A61K 31/66

(52) U.S. Cl. 514/114

(57) ABSTRACT

Methods and compositions are provided for preventing, reducing and reversing ischemic neuronal damage related to congenital and acquired ophthalmologic conditions such as macular degeneration, retinitis pigmentosa, optic neuritis, neuroretinitis, Lebers congenital amaurosis, Stargardts disease, Parkinson's disease, diabetic retinopathy, idiopathic senile vision loss, uveitis, edema and ocular surgery. An amount of an acetylcholine esterase inhibitor containing composition is administered to the eye of a mammal, either topically or via a controlled-release drug delivery system.

This application claims the benefit of U.S. Provisional Application No. 60/465,476 filed Apr. 25, 2003, which application is hereby incorporated by reference in its entirety.

### FIELD OF THE INVENTION

The present invention relates to a method and composition for treating and preventing ocular neuronal damage with periodic administrations of an acetylcholinesterase inhibitor. Specifically, the invention provides method and composition for treatment and prevention of congenital and acquired conditions (ischemic or non-ischemic) which threaten the nerves of the visual system of mammals; these conditions include but are not limited to: macular degeneration, retinitis pigmentosa, optic neuritis, neuroretinitis, Lebers congenital amaurosis, Stargardt disease, Parkinson's disease related vision loss, diabetic retinopathy, idiopathic senile vision loss, uveitis, edema and ocular surgery.

### BACKGROUND OF THE INVENTION

The health of a mammalian visual system is dependent upon the proper vascular perfusion of all constituent eye components, including: the retina, macula, choroid, sclera, ciliary body, conjunctiva and optic nerve. Afferent and efferent blood flow is critical to supplying nutrients, maintaining osmotic balances and removing waste products. The mammalian eye is vulnerable to many congenital and acquired focal ischemic conditions which can deprive the visual system of proper blood supply. Focal ischemia occurs under conditions in which a portion of the visual system is deprived of its normal blood supply, such as may result from choroidal neovascularization, the formation of drusen, reductions in ciliary activity, uveitis, edema, ocular surgery, traumatic injury, inherited diseases or visual pathway tumors. Focal ischemic conditions have the potential for producing widespread neuronal damage, even if the ischemic condition is transient. Much of this neuronal damage is attributed to secondary consequences of reperfusion of the tissue, such as the release of vasoactive products by damaged endothelium, and the release of cytotoxic products (free radicals, leukotrienes, etc.) by damaged tissues.

For decades, it has been demonstrated that within the mammalian visual nervous system, a phenomenon known as neuronal cell death takes place. This cell death is regulated by the release of neurotrophins. Neurotrophins are a family of small polypeptides, which bind to low affinity receptors throughout the visual system. Pereira, S. P. F., Araujo, E. G., 2000. Chronic Depolarization induced by veratridine increases the survival of rat retinal ganglion cells after 48 hours ‘in vitro’. Int. J. Dev. Neurosci. 18, 773-780.

Acetylcholine (ACh), the first neurotransmitter to be identified (Dale et al., 1936, Release of acetylcholine at voluntary motor nerve endings. J. Physiol. Lond. 82, 121-128) has recently been shown that an enhancement in ACh activity reduces neural cell death (Rinner, J., Kukulanky, T., Flesner, P., Skreiner, E., Globerson, A., Kasai, M., Hirokawa, K., Korsako, W., Schauenstein, K., 1994. Cholinergic stimulation modulates apoptosis and differentiation of murine thymocytes via A nicotinic effect on thymic epthelium. Biochem. Biophys. Res. Corn. 203, 1057-1062) and the death of related Purkinje cells (Mount et al., 1994, J. Neurochem. 63, 2065-2073).

The role of cholinergic activity in the differentiation and survival of retinal neurons is not well understood. It has been previously demonstrated that treatment with veratridine increases the survival of retinal ganglion cells. This effect was blocked by atropine indicating the importance of cholinergic activity on neuronal survival (Pereira et al., 2000, Int. J. Dev. Neurosci. 18, 773-780). Within the inner plexiform layer of the retina, muscarinic receptors have been identified on processes from all three inner retinal neuron types; in the outer plexiform layer, muscarinic receptors are critical to the functioning of second-order cells, with highest densities along the bipolar dendrites (Calaza et al., 2000, Brazirian J. Med & Biol Res. 33: 1075-1082). Niemeyer, et al., (1995) explored the impact of applying a muscarinic antagonist (Quinuclidinyl benzilate) to block of retinal cholinergic reception. They observed a dose-related decrease in retinal perfusion, suggesting a substantial contribution of muscarinic cholinergic transmission toward retinal viability (Niemeyer et al., 1995, Klin Monatsbl Augenheilkd., 206 (5): 380-383).

The healthy activity level of afferent cells such as rods and cones within the retina also plays an important role in regulating neuronal cell death. The blockade of electrical activity of afferent cells such as these will, in itself, induce neuronal degeneration within target cells. As cellular wastes build up between the RPE and Bruch's membrane, metabolic efficiency is disrupted and the overlying photoreceptors become ischemic and nonfunctional (Hageman et al., 2001, Prog Retin Eye Res., 20(6): 705-32).

These reports directly or indirectly focus on arriving at solutions to the regulation of neuronal activities and to the alleviation of visual disabilities. There is, however, a need for a simple and an effective method and composition for treatment and prevention of congenital and acquired ischemic conditions such as macular degeneration, retinitis pigmentosa, optic neuritis, neuroretinitis, Lebers congenital amaurosis, Stargardt disease, Parkinson's-related vision loss, diabetic retinopathy, solar retinopathy, retinal detachment, idiopathic senile vision loss, uveitis and edema. These conditions often threaten the nerves of the visual system of mammals.

In this regard, the U.S. Pat. No. 6,313,155 is of relevance because it discloses certain compositions and methods for increasing retinal blood flow and particularly for treating visual disabilities, such as macular diseases. In particular, it discloses that a topical carbonic anhydrase inhibitor in combination with an ocular hypotensive agent or inotropic agent either to the eye or systemically is effective to increase vascular perfusion and to treat macular edema and macular degeneration. It also reports that eye pressure reducing drugs or agents, when administered alone without a carbonic anhydrase inhibitor, tend to produce minimal changes in circulation and vision, and may in certain instances actually diminish both.

### SUMMARY OF THE INVENTION

In the present invention it is discovered, quite unexpectedly, that topical administration of acetylcholine esterase inhibitor to one or both eyes of the mammal affected by or vulnerable to ocular neuronal damage provides a profound beneficial effect (e.g., significant improvement in visual acuity) without any need for other inhibitors such as, for example, a topical carbonic anhydrase inhibitor. According to the present inventor's experience, the inclusion of carbonic anhydrase inhibitor into the topical composition was found to be unnecessary and associated with undesirable effects in achieving the goals of the present invention. It is believed that the acetylcholine esterase inhibitor causes increased ciliary activity, trabecular flow and choroidal perfusion within the mammalian eye and thereby significantly improving or at least stabilizing visual acuity.

Accordingly, the present invention provides a method and composition for preventing, reducing and reversing ocular neuronal damage related to various conditions (ischemic or non-ischemic conditions) affecting the visual system of a mammal. The composition has or consists essentially of an amount of an acetylcholine esterase inhibitor The present invention particularly provides a method of reducing neuronal damage related to an ischemic condition.

Specifically, the invention provides methods for preventing, reducing or reversing ocular neuronal damage and/or methods for improving visual acuity by topical administration of acetylcholine esterase inhibitor(s) to patients diagnosed with the following condition(s) and in need such therapeutic/prophylactic methods: macular degeneration, retinitis pigmentosa, optic neuritis, optic neuropathy, generalized optic nerve ischemia, neuroretinitis, Lebers congenital amaurosis, Stargardt disease, Parkinson's disease, diabetic retinopathy, idiopathic senile vision loss, uveitis, edema, ocular surgery, a thromboembolic event in the retinal vasculature, a visual scotoma, a retinal migraine, ophthalmoplegic migraine or scintillating scotoma, central retinal artery/vein occlusion, branch retinal artery/vein occlusion, anterior ischemic optic neuropathy, giant cell arteritis, retinal hemorrhage, cystoid macular edema, macular cystic degeneration, preretinal fibrosis, ischemic maculopathy, macular holes and cysts, macular epithelial fibrosis, peripapillary staphyloma and peripapillary atrophy, acute macular neuroretinopathy and/or Plaquenil-related toxicity.

In the present invention, it has been found that (2-mercaptoethyl)trimethylammonium iodide O,O-diethyl phosphorothioate sold as PHOSPOHLINE IODIDE® (also known as echothiophate) within its traditional dosage regimens reported in the prior art, does not exhibit the desired therapeutic effects described herein but it surprisingly is effective at very low concentrations, for example, of about 0.02% to 0.15% or even lower 0.01% to exhibit the desired therapeutic effects

### DETAILED DESCRIPTION OF THE INVENTION

The present invention relates to a method and composition for treating and preventing ischemic ocular neuronal damage and for improving visual acuity with periodic administrations of an acetylcholinesterase inhibitor. In particular, this invention utilizes the application of an ophthalmic acetylcholinesterase inhibitor, or pharmaceutical equivalent thereof, at low doses, to increase ocular Ach (acetyl choline) availability and thereby heighten muscarinic activity, ganglionic signal and retinal perfusion.

It is believed by the present inventor that the present treatment provides the desired therapeutic effects by amplification of synaptic transmissions through its enhancement of retinal ACh levels and muscarinic receptor functionality, thereby improving the quality of information destined for the occipital lobe of the brain. Specifically, the present inventor's unexpected success in reversing CNS-based visual loss related to, among other things, amblyopia, optic neuritis and Parkinson's disease has been disclosed. Furthermore, a muscarinic basis to the present effect is established here, through the induction of cycloplegic paralysis (using cyclopentolate). If induced on the morning immediately following treatment with low-dose echothiophate, one can observe no loss of subject vision gains, but if induced at day 4-5, there is significant, premature reversal of the effect.

The U.S. Pat. Nos. 6,273,092, 6,540,990 and 6,605,640, the contents of which are incorporated herein by reference, disclose details regarding treatment of various eye disorders using AChE inhibitor drugs.

Briefly, Acetylcholine esterase inhibitors are known to one skilled in the art. There are at least two AChE inhibitor drugs currently approved for clinical use on the eye in the United States. They are (2-mercaptoethyl) trimethylammonium iodide O,O-diethyl phosphorothioate sold as PHOSPOHLINE IODIDE® (Wyeth-Ayerst, Philadelphia, Pa.), and physostigmine (also known as eserine) sold as ANTILRIM® (Forest Pharmaceuticals, St. Louis, Mo.). PHOSPHOLINE IODIDE is dispensed as eyedrops at a desired potency. PHOSPHOLINE IODIDE of various concentrations, such as for example 0.25%, 0.125%, 0.06% and 0.03% and a pharmaceutically acceptable sterile diluent to dilute the concentrated form of this drug are commercially available. PHOSPHOLINE IODIDE is currently used for glaucoma and accommodative esotropia. As such, PHOSPHOLINE IODIDE is not a preferred drug even to treat glaucoma and accommodative esotropia because of many adverse side effects caused by this drug when it is used in the current regimen of multiple times a day at high concentrations. Some of the side effects known to be caused by the currently recommended doses of this drug (for glaucoma at 0.12 and 0.25 BID) are iris cysts, cataract formation especially anterior subcapsular, posterior synechiae and elevated intraocular pressure.

In the present method, the cholinesterase inhibitor, such as phospholine iodide, administered in concentrations many fold more dilute than currently available pharmacological preparations, applied to the eye before sleep will achieve alleviation of the deteriorated or deteriorating vision with none of the unacceptable side effects of the usual pharmacological preparations and without the loss of peripheral vision. The effect of one administration of the inhibitor can last for many days. The present invention shows that the effective concentration of AChE inhibitor in the composition to treat diseases associated with the posterior region of the eye can be very low (for example, as low as at least 0.001% to about 0.0075% of PHOSPHOLINE IODIDE) to be effective. The invention discloses that such a concentration is extremely useful medically. Specifically, this lower dose range is especially useful in providing eye drugs that will contain a concentration of AChE inhibitor that is low enough to be both safe and effective. For example, one application of a drop of a suitable composition containing 0.03% PHOSPHOLIE IODIDE is sufficient for few days.

The composition administered to the eye should have a pharmaceutically acceptable carrier and a selected AChE inhibitor suspended or dissolved in the carrier. The concentration of AChE inhibitor in the composition administered to the eye and the method of administration of the composition in accordance with this invention depends on the type of AChE inhibitor containing composition used for therapy. For example, preferred concentrations of PHOSPHOLINE IODIDE in the PHOSPHOLINE IODIDE containing composition are from about 0.25% to about 0.001%. More preferred PHOSPHOLINE IODIDE concentrations are from about 0.15% to about 0.005%. Most preferred PHOSPHOLINE IODIDE concentrations are about 0.12%, 0.03% and 0.0075%. Still more preferred concentrations are about 0.01%, 0.015% and 0.02%. It is preferred to apply PHOSOSPLIE IODIDE topically to the eyes in the form of eye drops. Although it is preferred that these solutions with various concentrations of PHOSPHOLINE IODIDE are stored in a refrigerator, they an be stored at room temperature for about two months or even beyond two months without losing their efficacy to restore near vision in presbyopic patients.

A solution containing chlorobutanol (0.55%), mannitol (1.2%) boric acid (0.6%) and exsiccated sodium phosphate (0.026%) can be used as a carrier solution and/or as a diluent for PHOSPHOLINE IODIDE. While this solution is presently sold as a diluent in the kit containing PHOSPHOLINE IODIDE, other pharmaceutically acceptable carriers or excipients that are known to enhance membrane permeability and cellular uptake of the drug can be used as diluents with or without modification for application to the eye. Such carriers are known to one skilled in the art.

In a preferred embodiment of the invention, the AChE inhibitor is administered at bedtime. A single topical application of a given AChE inhibitor at bedtime can enhance visual acuity in the phakic emmetropic patients as well as in pseudophakic patients for a few days. For example, application of one to two drops of PHOSPHOLINE IODIDE of a selected concentration at bedtime can alleviate the diminished vision of the patients for at least five days. Preferably, the following steps are followed every time AChE inhibitor is applied to the patient. The first step is to read for about 30 minutes. The second step is to administer an AChE inhibitor of a selected concentration. The third step is to sleep. Without wishing to be bound by any theory or explanation, it is believed that the reading for about 30 minutes preconditions eye muscles and visual pathway to respond better to the AChE inhibitor treatments. It takes about 6 to 8 hours of sleep to notice the restoration. If one is awaken in the middle of sleep, the individual may notice partial effect but after 6 to 8 hours of sleep the effect will be maximized. By the term “bedtime” it is meant that the time when the patient goes to sleep for about 6 to 8 hours, regardless of whether it is during the day or night time. The composition is administered at bedtime, i.e., a drop of the AChE inhibitor is administered just before the patient goes to sleep for about 6 to 8 hours.

In another preferred embodiment of the invention, the AChE inhibitor is administered prior to sleep, i.e., a drop of the AChE inhibitor is administered immediately before the patient closes his/her eyes for at least four hours of continuous sleep. It is important that the patient does not awaken or open their eyes after taking the drop, as such activity will cause the drops to be cleared from the surface of the eye via the tear ducts.

Accordingly, by practicing the present invention, one can achieve a definite, measurable gain in visual acuity in patients with retinal vascular or choroidal vascular disease or other diseases or conditions of the eye when the one is administered with the acetylcholinesterase inhibitor, in the dilution and the manner outlined above. Increase in visual acuity can be measured by techniques well known to those skilled in the art. Although the mechanism of action is unknown, it is believed that a suitable dose of AChE inhibitor administered at “bedtime” or “prior to sleep,” as defined herein, may allow the eye to accumulate sufficient stockpiles of acetylcholine by inhibiting acetylcholine esterase activity in the eye and strengthen the eye muscles leading to the normal perfusion of the blood to the posterior region of the eyeball particularly choroid blood vessels. Retinal and choroidal function and health are dependant on normal perfusion of these tissues.

Choroidal circulation and retinal perfusion are visibly increased, within the effects of low-dose echothiophate. This is supported by before and after fluorescent angiograms performed across trial subjects. Additionally, increased ciliary body activity increases blood flow to and from the choroid.

Although ophthalmic compositions containing acetylcholinesterase inhibitors are known in the art (see, Cohen, 1966, American Journal of Ophthalmology, 62:303-312 and Physician's Desk Reference for Ophthalnic medicines, 2001 (29^{th} edition), pp 321-323), it has been found that within their traditional dosage regimens, these compositions do not exhibit the therapeutic effects desired herein. Further, these existing compositions typically have to be applied two to three times a day. It has been found that such repeated administration is not optimal in practice, because, inter alia, for optimal treatment the patient has to have the medicament always available and the patient is disturbed several times a day. Such multiple administration of a drug, in particular of an ophthalmic composition, leads generally to the problem of overdosing and underdosing.

Surprisingly, it has now been found that an ophthalmic acetylcholinesterase inhibitor such as Phospholine Iodide (ecothiophate) can be formulated for weekly or bi-weekly administration at low-dose, which administration provides therapeutic efficacy in the eye over about 7 days and that such compositions are surprisingly well tolerated. Moreover the above-mentioned bi-weekly or weekly ophthalmic compositions produce a highly reliable and more beneficial clinical result in a patient treated therewith.

Therefore, in one aspect the present invention provides an ophthalmic composition suitable for weekly administration to the eye before sleep. The composition has an AChE inhibitor from about 0.001-0.25%. Preferred inhibitor is (2-mercaptoethyl) trimethylammonium iodide O,O-diethyl phosphorothioate. Preferred concentrations of the inhibitor is 0.010%, 0.015% and 0.020%. In one aspect of the invention, the concentration of the inhibitor does not exceed 0.025% and is used only for weekly administration. The low-dose echothiophate as referred to herein is that composition which has the ecothiophate at a concentration less than 0.03% and/or is applied no more than twice a week at the rate of one drop per each application. Thus, the low-dose echothiophate can be acheived by adjusting the solution strength (e.g., 0.001-0.025%) and/or modifying the frequency of administration (bi-weekly or once a week). It is preferred to adjust the solution strength rather than modifying the frequency of administration. Such The application of a long-acting cholinesterase inhibitor (AChE), such as ECHO, permanently blocks the binding sites of existing cholinesterase enzymes, halting their breakdown of acetylcholine. Levels of acetylcholine remain artificially elevated until the body naturally replaces these inactivated cholinesterase enzymes (3-5 days) (Pappano A. J., 1998 Cholinoceptor-Activating & Cholinesterase-Inhibiting Drugs, In Basic and Clinical Pharmacology, 7th Edition, (Katzung, B. G., ed) Appleton & Lange, pp. 93-94). One indicative effect of this solution is a strengthening of ciliary-based accommodative potential. An increase in accommodative amplitude can be measured for 4-6 days.

A drop of the compositions of the present invention amounts to about 10-100 μl (microliters), preferably about 20-70 μl, and especially about 25-50 μl and more preferably about 30 μl. It is preferred that drops are applied inside the patient's lower eyelid. While administering the drop, patient may PINCH the bridge of their nose to block drainage into the tear ducts, then to continue compressing the tear ducts for two minutes post-application. Instead of drops, the composition may also be designed as controlled release forms, a dermal patch for application on the surface of the eyelids or in the form of a collagen lens for laying over the eye to be treated at bedtime or prior to sleep.

Mammals in the present invention include not only humans but also other animals selected from a group consisting of mice, rats, rabbits, pigs, cows, goats, dogs, cats and monkeys.

All publication references, patents and patent applications mentioned in this specification are indicative of the level of those skilled in the art to which this invention pertains. The contents of all the publications, patents and patent applications are herein incorporated by reference to the same extent as if each individual publication, patent or patent application was specifically and individually indicated to be incorporated by reference.

### EXAMPLES

The examples below are carried out using standard drug administration techniques, that are well known and routine to those of skill in the art, except where otherwise described in detail. The examples are illustrative, but does not limit the invention. These examples illustrate among other things (1) the alleviation of diminished visual acuity in humans suffering from diseases or disorders of the anterior and/or posterior segment(s) of the eye and (2) potentiation of baseline normal vision by topical administration of an AChE inhibitor to the eye.

Thirty three patients were studied with ages 11-76 Etiology varied from amblyopia to Stargardt diseases. All of the patients studied showed visual acity improvments after low-dose AchE inhibitor, PHOSPHOLINE IODIDE (also referred to herein as ECHO), treatment. Medications were applied prior to sleep. The dosage regimen was such that patients will begin therapy using the opthalinc composition containing the AchE inhibitor at a concentration of 0.015% applied to one eye on Sunday evening and the alternate eye every Wednesday evening. Thereby, each eye received one drop per week. Most patients realized visual acuity improvements with a solution of 0.015% strength. During the course of treatment, it was found that in select cases, this 0.015% dosage was not well tolerated by older patients and pseudophakic patients. A lower dosage of 0.010% was then introduced.

Conversely it was found that young eyes (age 11-15) appeared to show reduced absorption of the 0.015% solution, relative to the older patients. Those patients, who displayed this reduced absorption (as evidenced by a lack of pupillary constriction) the dosages were increased to 0.020%. Drops were applied inside the patient's lower eyelid. The patients were advised to apply drops just before the patient closes his/her eyes and have at least for three to four hours of continuous sleep. Patients were advised to not awaken or open their eyes after taking the drop, as such activity will cause the drops to be cleared from the surface of the eye via the tear ducts.] While administering the drop, patients were advised to pinch the bridge of their nose to block drainage into the tear ducts, then to continue compressing the tear ducts for two minutes post-application.

All patients were given pre treatment comprehensive examinations and patients monitored their visions during the treatment period. The improvement in visual acuity was immediate and generally noticed on the first day or week of treatment. The patient treatment results are presented in table below.

TABLE ONE
Examples of visual acuity improvements within low-dose echothiophate human subjects.

| | | | | | | Near Vision | | Color Vision | |
|---|---|---|---|---|---|---|---|---|---|
| | | | | Distant Vision | | (Jaeger) | | (Ishihara) | |
| Subject | | | | Pre- | Post- | Pre- | Post- | Pre- | Post- |
| Initials | Sex | Condition | Dosage | ECHO | ECHO | ECHO | ECHO | ECHO | ECHO |
| BB2 | F | Amblyopia | 0.010 | 20/200 | 20/200 | 18 | 16⁻¹⁺¹ | 0 | 5 |
| NM | M | Amblyopia | 0.015 | 20/70 | 20/50⁺² | 1 | 1 | 10 | 10 |
| WD2 | F | Amblyopia | 0.010 | 20/50²⁺³ | 20/30⁻² | 7 | 2 | 10 | 10 |
| DK | M | Brain Tumor | 0.015 | 20/70 | 20/70⁺² | 3 | 1⁺ | 8 | 10 |
| BB | F | Cerebral Stroke | 0.015 | 20/50 | 20/40⁻¹ | 7⁻ | 2⁻¹ | 1 | 1 |
| BH | M | Central Serous | 0.015 | 20/300 | 20/70⁻ | 16 | 1⁻² | 2 | 8 |
| | | Chorioretinopathy | | | | | | | |
| AV | M | Diab. Retinopathy | 0.015 | 20/70 | 20/30⁻¹ | 7⁺ | 3⁺ | N/A | N/A |
| BR | F | Diab. Retinopathy | 0.010 | 20/1600 | 20/1600 | 18 | 18⁻¹ | 0 | 0 |
| EM | M | Diab. Retinopathy | 0.010 | 20/25⁻ | 20/20⁻ | 1⁻¹ | 1 | 10 | 10 |
| TY | M | Diab. Retinopathy | 0.010 | 20/50⁻ | 20/50⁺ | 16 | 16 | N/A | N/A |
| CO | F | Macular Hole | 0.015 | 20/100⁻ | 20/70⁻¹ | 16 | 3⁻ | N/A | N/A |
| TO | M | Macular Hole | 0.015 | 20/1600 | 20/200 | 18⁺ | 10 | N/A | N/A |
| KC | F | Migraine/Amblyopia | 0.015 | 20/8000 | 20/1600 | 100 | 54 | 2 | 8 |
| JJ | F | Optic Neuritis | 0.015 | 20/100⁻ | 20/25⁺ | 5 | 1⁺ | 10 | 10 |
| MM | M | Optic Neuritis | 0.015 | 20/40 | 20/25⁺ | 3⁻ | 1 | 0 | 0 |
| HN | M | Parkinson's | 0.015 | 20/40 | 20/20⁺² | 3 | 1 | 10 | 10 |
| BC | F | Photocoagulation | 0.010 | 20/70 + 2 | 20/70 + 3 | 5 − 1 | 3 − 2 | 7 | 8 |
| PB | M | Photocoagulation | 0.010 | 20/40 | 20/25 | 2+ | 1 +− 1 | 10 | 10 |
| RD2 | M | Photocoagulation | 0.015 | 20/1600 | 20/400 | 20 | 18 | 0 | 0 |
| VC | F | Photocoagulation | 0.010 | 20/2667 | 20/400 | 20/800 | 16 | 0 | 1 |
| CR | F | Preretinal Fibrosis | 0.015 | 20/30⁻¹ | 20/25⁺¹ | 1⁻¹ | 1⁻ | 10 | 10 |
| TL | M | Retinal Detachment | 0.010 | 20/25⁻ | 20/20⁻¹ | 5 | 5 | 10 | 10 |
| VD | F | Retinal Hole | 0.015 | 20/100 | 20/100⁺⁺ | 16 | 3⁺² | 10 | 10 |
| SB | F | Retinal Vein | 0.010 | 20/1600 | 20/1000 | 16 | 16⁺ | 2 | 8 |
| | | Occlusion | | | | | | | |
| KH2 | F | Retinitis Pigmentosa | 0.015 | 20/400 | 20/70⁻¹ | 7⁻ | 2 | 0 | N/A |
| ED | M | Retinitis Pigmentosa | 0.015 | 20/8000 | 20/70 | 16 | 7 | 0 | 8 |
| RH2 | M | Retinitis Pigmentosa | 0.015 | 20/4000 | 20/2000 | 16 | 10 | 0 | 0 |
| VD2 | M | Retinitis Pigmentosa | 0.015 | 20/30⁻³ | 20/25⁺⁴⁻² | 7 | 1⁺ | 5 | 8.5 |
| SL | M | Solar Retinopathy | 0.010 | 20/30⁻¹ | 20/25 | N/A | N/A | N/A | N/A |
| AF | F | Stargardts | 0.015 | 20/1600 | 20/200 | 5″/J2 | 1 | 0 | 10 |
| AG | M | Stargardts | 0.015 | 20/300 | 20/100⁻¹ | 10 | 1⁻ | 7 | 10 |
| GP | M | Stargardts | 0.015 | 20/300 | 20/400 | 3″/J2 | 6″/J2 | N/A | N/A |
| KH | F | Stargardts | 0.015 | 20/200⁺¹ | 20/100⁻¹⁺³ | 5⁻ | 1 | 10 | 10 |

Although the foregoing invention has been described in some detail by way of illustration and example for purposes of clarity of understanding, it will be obvious that certain changes and modifications may be practiced within the scope of the appended claims.

What is claimed is:

1. A method of preventing, reducing and reversing ocular neuronal damage related to conditions affecting the visual system of a mammal, comprising: administration to one or both eyes of a mammal affected by or vulnerable to ischemic ocular neuronal damage, an amount of a acetylcholine esterase inhibitor containing composition sufficient to provide a therapeutic benefit.

2. The method of claim 1, wherein the composition is administered immediately prior to sleep.

3. The method of claim 2, wherein said inhibitor is (2-mercaptoethyl)methylammonium iodide O,O-diethyl phosphorothioate.

4. The method of claim 3, wherein said (2-mercaptoethyl)trimethylammonium iodide O,O-diethyl phosphorothioate is present at a concentration of 0.001% to 0.25%.

5. The method of claim 2, wherein the acetylcholine esterase inhibitor is contained in a pharmaceutically acceptable buffer medium.

6. The method of claim 1, wherein the ocular neuronal damage relates to at least one condition or disorder selected from the group consisting of: macular degeneration, retinitis pigmentosa, optic neuritis, optic neuropathy, generalized optic nerve ischemia, neuroretinitis, Lebers congenital amaurosis, Stargardt disease, Parkinson's disease, diabetic retinopathy, idiopathic senile vision loss, uveitis, edema, ocular surgery, a thromboembolic event in the retinal vasculature, a visual scotoma, a retinal migraine, ophthalmoplegic migraine or scintillating scotoma, central retinal artery/vein occlusion, branch retinal artery/vein occlusion, anterior ischemic optic neuropathy, giant cell arteritis, retinal hemorrhage, cystoid macular edema, macular cystic degeneration, preretinal fibrosis, ischemic maculopathy, macular holes and cysts, macular epithelial fibrosis, peripapillary staphyloma and peripapillary atrophy, acute macular neuroretinopathy and/or Plaquenil-related toxicity.

7. A method of improving visual acuity in a patient in need thereof and suffering from at least one condition or disorder selected from the group consisting of: amblyopia, brain tumor, cerebral stroke, central serous chorioretinopathy, diabetic retinopathy, macular hole, retinal migraine, scintillating scotoma, optic neuritis, Parkinson's disease, photocoagulation, preretinal fibrosis, retinal detachment, retinal hole, retinal vein occlusion, retinitis pigmentosa, solar retinopathy and Stargardt disease.

8. The method of claim 7, wherein the composition is administered prior to sleep.

9. The method of claim 8, wherein said inhibitor is (2-mercaptoethyl)trimethylammonium iodide O,O-diethyl phosphorothioate.

10. The method of claim 9, wherein said (2-mercaptoethyl)trimethylammonium iodide O,O-diethyl phosphorothioate is present at a concentration of 0.001% to 0.25%.

11. The method of claim 10, wherein the concentration of (2-mercaptoethyl)trimethylammonium iodide O,O-diethyl phosphorothioate is 0.001%.

12. The method of claim 10, wherein the concentration of (2-mercaptoethyl)trimethylammonium iodide O,O-diethyl phosphorothioate is 0.0075%.

13. The method of claim 10, wherein the concentration of (2-mercaptoethyl)trimethylammonium iodide O,O-diethyl phosphorothioate is 0.010%.

14. The method of claim 10, wherein the concentration of (2-mercaptoethyl)trimethylammonium iodide O,O-diethyl phosphorothioate is 0.015%.

15. The method of claim 10, wherein the concentration of (2-mercaptoethyl)trimethylammonium iodide O,O-diethyl phosphorothioate is 0.02%.

16. The method of claim 10, wherein the concentration of (2-mercaptoethyl)trimethylammonium iodide O,O-diethyl phosphorothioate is 0.03%.

17. An ophthalmic composition comprising an acetylcholinesterase inhibitor in an ophthalmic buffer solution, wherein the acetylcholinesterase inhibitor is present at a concentration of less than 0.03%.

18. The composition of claim 17, wherein the composition is administered once weekly, prior to sleep.

19. The composition of claim 17, wherein said inhibitor is (2-mercaptoethyl)trimethylammonium iodide O,O-diethyl phosphorothioate.

20. The composition of claim 17, wherein said (2-mercaptoethyl)trimethylammonium iodide O,O-diethyl phosphorothioate is present in said composition at a concentration of about 0.015%.
